(19) United States
(12) Patent Application Publication
(10) Pub. No.: US 2004/0143216 A1
(43) Pub. Date: Jul. 22, 2004

(54) Adapter connector for an infusion set and inserter system

(75) Inventors: Joel S. Douglas, Groton, CT (US); Stephanie S. Anderson, Atlanta, GA (US); Keith D. Ignotz, Duluth, GA (US); Robert Rosenzweig, Atlanta, GA (US)

Correspondence Address: BURNS DOANE SWECKER & MATHIS L L P, POST OFFICE BOX 1404, ALEXANDRIA, VA, 22313-1404, US

(73) Assignee: SpectRX, Inc.

(21) Appl. No.: 10/654,272

(22) Filed: Sep. 2, 2003

Related U.S. Application Data
(60) Provisional application No. 60/407,531, filed on Aug. 30, 2002.
(60) Provisional application No. 60/409,119, filed on Sep. 9, 2002.
(60) Provisional application No. 60/409,120, filed on Sep. 9, 2002.

(51) Int. Cl. A61M 5/00

(52) U.S. Cl. 604/116

(57) ABSTRACT

An adapter is provided for use with an infusion set and inserter device used for placement of a subcutaneous infusion set or the like used for delivering a selected medication to a patient. The adapter comprises a first end adapted to be removably coupled to the infusion set and a second end adapted to be removably coupled to an insertion device, particularly the sled fixtured in a track of the insertion tool. The adapter is designed for receiving and supporting an infusion set in a position in the inserter which is used for placement of and insertion of the needle or cannula through the skin of a patient at a selected insertion site. The provided adapter connectors enable the use of a variety of insertion sets having differing configurations with a given inserter having a given configuration.

### FIELD OF THE INVENTION

This invention relates to infusion set and inserter systems that are used to provide a patient with a quick and painless way to place in the patient's body a subcutaneous infusion needle or cannula for medication administration, such as insulin.

### BACKGROUND OF THE INVENTION

This invention relates generally to insertion set and inserter device systems used to provide automatic placement of a medical infusion device, including a needle or cannula inserted through the skin of a patient for subcutaneous infusion of medication. More specifically, this invention relates to a compact and easily operated injector or inserter device for placement of an infusion needle or cannula of a subcutaneous infusion set or the like through the skin of a patient with a controlled force and insertion speed, and particularly wherein the injector or inserter is adapted for use by the patient. Typically such infusion systems employ a two-part insertion set, an inserter and a tubing element for administration of the medication from the container to the inserted infusion needle or cannula. In use the patient places the two-part insertion set in the inserter. The inserter propels the insertion set at various angles from 5° to 90° (some vertically at 90° and some at an angle of 20° to 45° to the skin) against the patient's skin to subcutaneously insert the infusion needle or cannula. The inserter is removed. Then the outer piece of the insertion set is removed leaving the base piece of the insertion set attached to the skin. The tubing element is then placed in or connected to the base piece where the outer piece was removed to provide administration of the medication from a container to and through the subcutaneous needle or cannula of the infusion set.

Medical needles are widely used in the course of patient care and treatment, particularly with respect to the delivery of selected medications to a patient. In one common form, hollow hypodermic needles or soft cannula are employed for transcutaneous delivery of a selected medication from a syringe, pump or the like. In another common form, insertion needles are employed for transcutaneous placement of a soft and relatively flexible tubular cannula by use of an insertion needle, followed by insertion needle removal and subsequent infusion of medical fluid to the patient through the cannula. The inserter inserts the insertion set containing the infusion needle and/or cannula using a spring-loaded advancement with a controlled force and speed toward the patient's skin to transcutaneously place the infusion needle and/or cannula. When the plunger head reaches a fully advanced position, with the infusion set placed on the patient, the infusion set is releasable from the inserter with minimal force to permit quick and easy separation of the set.

In certain medical treatment regimens, it may be necessary or desirable for the patient to transcutaneously place the medical needle. For example, diabetic patients frequently self-administer insulin injections or periodically place a subcutaneous infusion set for subsequent programmable delivery of insulin by means of a medication infusion pump or manual infusion device as described in the copending application 60/170,570 and related U.S. Pat. No. 6,482,186 for reusable injection delivery device using a dual mode injection system of a catheter or insulin pen tip for injection of medication. The infusion pumps are of the general type described in U.S. Pat. No. 4,685,903. Such subcutaneous infusion sets along with insertion sets and corresponding insertion deices or inserters are disclosed, for example, in U.S. Pat. Nos. 4,755,173; 5,176,662; 5,257,980; 6,056,718; 6,293,925 and in U.S. Patent Application Publication No. 2002/0022855. The disclosures of the above patents and applications are hereby incorporated herein by reference in their entirety.

Some patients are reluctant or hesitant to pierce their own skin with a medical needle, and thus encounter difficulties in correct needle placement for proper administration of the medication. Such difficulties can be attributable to insufficient manual dexterity or skill to achieve proper needle placement or alternately to anxiety associated with anticipated discomfort as the needle pierces the skin. This problem can be especially significant with medications delivered via a subcutaneous infusion set, since incorrect placement can cause kinking of the cannula and resultant obstruction of medication flow to the patient. Cannula kinking can be due to infusion set placement at an incorrect angle relative to the patient's skin, and/or needle placement with an incorrect force and speed of insertion.

Automatic injectors or inserter devices have been used to provide assistance in correct placement of insertion sets including inserting the needles or cannulas of infusion sets, for example for use with insulin syringes by patients with diabetes. These inserter device/insertion set systems help the patient overcome the fears or dexterity problems associated with inserting the infusion set needles or cannulas into their skin. However, the inserters do not work for all insertion sets and there is a need for patients to use a variety of sets with a single inserter to achieve comfortable use of the infusion set of their choice. The configuration and manual operation of the insertion set may determine the patient's choice of insertion set for preferred use. For example, the patient will typically purchase an inserter or insertion device with one or more insertion sets compatible for use with the inserter. A typical such system comprises the Medtronic MiniMed “Sil-Serter” inserter device and the “Silhouette” insertion set, as depicted in the above-referenced U.S. Patent Application Publication No. 2002/0022855, particularly FIG. 51 therein, which illustrates the inserter intended for use at a low angle (20-45°) to the skin; vertical or 90° inserter systems are also illustrated. (“Sil-Serter” and “Silhouette” are trademarks of Medtronic MiniMed.) As can be seen in that example, the latching means holding the two parts of the insertion set together are located distal or remote from the inserter device when inserted therein. Also, the gripping means for manually releasing the latching means is co-located with the latching means distal or remote from the inserter device and is part of the outer piece of the insertion set that is to be removed after the insertion set is placed on the patient's skin by the action of the inserter. This means that after the inserter device is removed, and it is desired to remove the outer piece of the insertion set, leaving the base piece in contact with the patient's skin with the needle or cannula inserted subcutaneously, the latching release means must be gripped by the patient close to the base piece on the skin and, while holding the base piece in place, pull the outer piece out of the base piece. For some patients, this is difficult because of the close proximity of the gripping means that must be pulled away to the base piece that stays in place and because of the dexterity required, which those patients cannot manage or have difficulty managing.

As a consequence of the above, some patients would prefer to have an insertion set that is easier to use and that requires less dexterity to manipulate. Such an insertion set with a different configuration is provided by SpectRx, Inc. as the “Simple Choice” (trademark) insertion set, which has the latching means proximal to the inserter and has the latching means and gripping means built in as part of the base piece that remains on the patient's skin when the outer piece of the insertion set is removed. This type of insertion set is disclosed in copending U.S. patent application Ser. No. 09/675,159, which has published as PCT International Patent Application No. WO 01/052617 (See FIG. 5 therein.), the disclosure of which is incorporated herein by reference in its entirety. The gripping means for manually releasing the latching means is located distal or remote from the inserter device, but is also part of the base piece of the insertion set that remains on the patient's skin. In this instance, after the inserter device is removed, and it is desired to remove the outer piece of the insertion set, leaving the base piece in contact with the patient's skin with the needle or cannula inserted subcutaneously, the latching release means, i.e., the gripping means on the base piece, is gripped by the patient to simultaneously release the outer piece and hold the base piece in place on the skin. The outer piece of this type of insertion set is then easily removed, requiring less dexterity than the above insertion set product where the gripping means is on and part of the outer piece being removed.

The patient typically wants to have one inserter device and several disposable insertion sets available for use. The problem is that the above different configuration insertion set with the proximal latching means will not fit into and is incompatible with the inserter that is adapted to accept the insertion set having the distal locking means. Thus, it is apparent that the patient desires to be able to use different configuration insertion sets with a single inserter device, which is not possible. Thus, there is a need for improved flexibility in consumer product usage capabilities.

### SUMMARY OF THE INVENTION

The present invention relates to an adapter connector to be used with an automatic injector, particularly for use with a subcutaneous infusion set, for quickly and easily placing the infusion through the skin of a patient at the correct insertion angle.

In accordance with the invention, an adapter used with an injector or inserter is provided for quick and easy transcutaneous placement of a medical needle through the skin of a patient, particularly such as an insertion needle and related cannula of a subcutaneous infusion set. The adapter is designed to mate with the infusion set and with the injector so as to place the needle through the skin at a selected insertion angle and with a controlled force and speed of insertion, to insure proper needle placement with minimal patient discomfort. The adapter for the injector or inserter is particularly designed to meet these objectives and insure insertion of the needle with a minimum of pain.

An adapter is provided for use with an infusion set and inserter device used for placement of a subcutaneous infusion set or the like used for delivering a selected medication to a patient. The adapter comprises a first end adapted to be removably coupled to the infusion set and a second end adapted to be removably coupled to an insertion device, particularly the sled fixtured in a track of the insertion tool. The adapter is designed for receiving and supporting an infusion set in a position in the inserter which is used for placement of and insertion of the needle or cannula through the skin of a patient at a selected insertion site. The provided adapter connectors enable the use of a variety of insertion sets having differing configurations with a given inserter having a given configuration.

The adapter connector of this invention comprises a fixture with the proximal end being configured to removably work with the injector sled and the distal end being removably attachable to an infusion set. The injector (inserter) has a spring-loaded or pneumatic plunger attached to a sled in a track design to guide the infusion set needle and plunger to the targeted skin section. The adapter is designed for retention of the infusion set within a sled end of the injector and the infusion set.

In one aspect, this invention provides an adapter connector for use in an insertion set and inserter system comprising a first end for connecting with the inserter and a second end for connecting with the insertion set wherein the first end of the connector is adapted to fit into and connect with the portion of the inserter device configured for receiving a two part insertion set having side positioned releasable latching means for holding the two parts of the insertion set together which latching means is located distal from the inserter device when the insertion set is positioned in the inserter device; and the second end of the connector is adapted to receive a two-part insertion set having incompatible size or shape for fitting into the portion of the inserter device configured for receiving an insertion set and having side positioned releasable latching means for holding the two parts of the insertion set together which latching means is positioned proximal to the connector when the insertion set is positioned in the connector; whereby the connector is adapted to connect the insertion set and the inserter, thereby enabling the use of the insertion set with the inserter device where the insertion set is otherwise incompatible with or unusable in said inserter device.

In another aspect, this invention provides a method for subcutaneous placement of an infusion needle or cannula with a system comprising an insertion set and an inserter device comprising the steps of selecting an inserter device adapted for placement of an insertion set comprising an infusion needle or cannula; selecting an insertion set that is incompatible with said inserter; selecting an adapter connector having a first end adapted to fit into and connect with the portion of the inserter configured for receiving an insertion set and having a second end adapted to receive said insertion set that is incompatible by size or shape with said inserter device.

In another aspect this invention provides a system for subcutaneous placement of an infusion needle or cannula comprising an inserter device adapted for subcutaneous placement of an infusion needle or cannula by placing an insertion set containing said needle or cannula against the surface of the skin; an insertion set comprising an infusion needle or cannula adapted for subcutaneous placement having a size or shape incompatible for fitting into the inserter device; and an adapter connector comprising a first end adapted to fit into the inserter device and a second end adapted to receive the insertion set; whereby the adapter connector is adapted to connect the insertion set and the inserter device, thereby enabling the use of the insertion set with the inserter device where the insertion set is otherwise incompatible with or unusable in said inserter device.

In another aspect this invention provides An adapter connector for use in an insertion set and inserter system comprising a first end for connecting with the inserter and a second end for connecting with the insertion set wherein the first end of the adapter connector is adapted to fit into and connect with the inserter device configured for receiving an insertion set comprising an infusion needle or cannula adapted for subcutaneous placement and the second end of the adapter connector is adapted to receive an insertion set having incompatible size or shape for fitting into the inserter device; whereby the adapter connector is adapted to connect the insertion set and the inserter, thereby enabling the use of the insertion set with the inserter device where the insertion set is otherwise incompatible with or unusable in said inserter device.

Other features and advantages of the present invention will become more apparent from the following detailed description, taken in conjunction with the accompanying drawings that illustrate, by way of example, the principle of the invention.

### BRIEF DESCRIPTION OF THE DRAWINGS

The accompanying drawings illustrate the invention. In such drawings:

FIG. 1 is an elevation view of an inserter;

FIG. 2 is an isometric view illustrating an embodiment of the invention;

FIG. 2A is an isometric view illustrating an embodiment of the invention;

FIG. 3 is an isometric view illustrating an embodiment of the invention;

FIG. 4 is an isometric view illustrating an embodiment of the invention;

FIG. 5 is an isometric view illustrating an embodiment of the invention;

FIG. 6 is an isometric view illustrating an embodiment of the invention;

FIG. 7 is an isometric view illustrating an embodiment of the invention;

FIG. 8 is an elevation view of an embodiment of the invention;

FIG. 9 is an elevation view of an embodiment of the invention;

FIG. 10 is a top view of an embodiment of the invention;

FIG. 11 is a section elevation view of an embodiment of the invention;

FIG. 12 is a section elevation view of an embodiment of the invention;

FIG. 13 is an exploded isometric view of an embodiment of the invention.

### DETAILED DESCRIPTION OF THE INVENTION

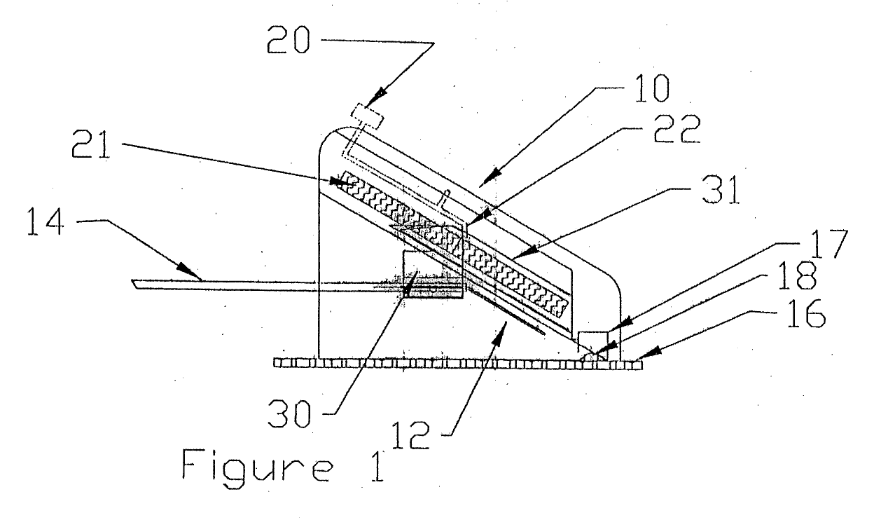

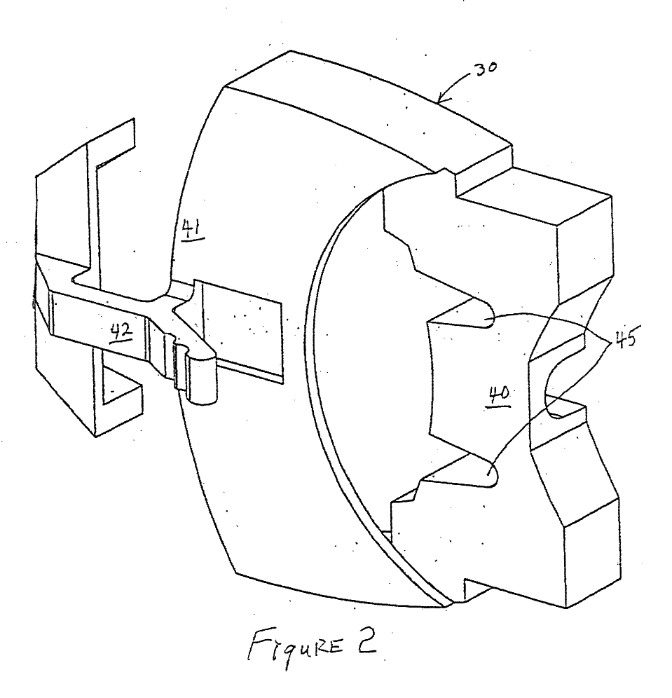

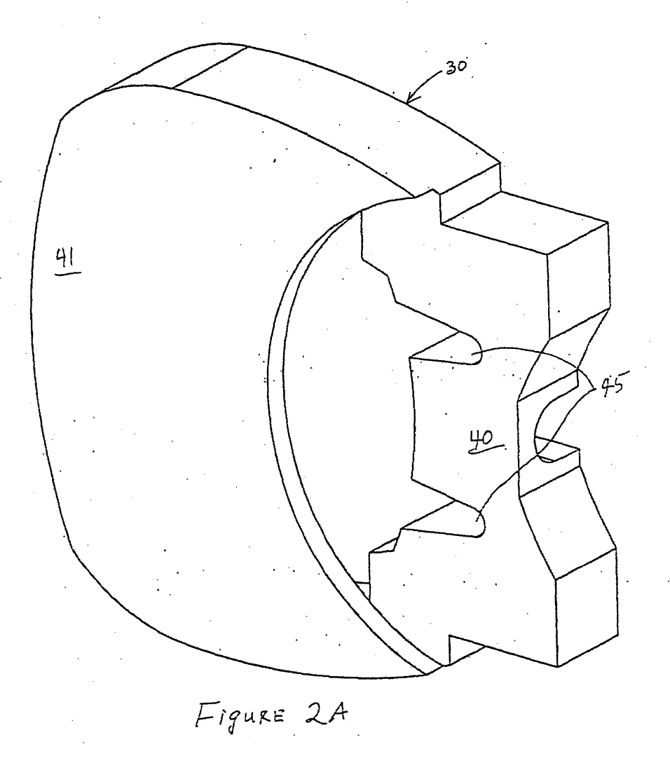

As shown in FIG. 1, an adapter referred to generally by the reference numeral 30 is provided for quick and easy attachment to an infusion set 14 in an injector 10 for transcutaneous placement of a medical cannula or needle 12, particularly such as with an subcutaneous infusion set. The adapter 30 illustrated in one embodiment in FIG. 2, includes a proximal end 40 and distal end 41, and injector 10 includes latch release mechanism 20.

The adapter 30 is loaded into the injector such that it is held by the adapters gripping mechanism so that the proximal end 40 is in communication with the injector. The patient then presses on the latch 42 on the distal end 41 of the adapter 10 and loads an infusion set into the distal end 41 of adapter 30.

Adapter 30 can be made of soft material such as silicone, metal, plastic such as polycarbonate, or other material appropriate to the system in which it is used.

The automatic injector 10 as shown in the illustrative drawing FIG. 1, is particularly designed for placement of the insertion needle 12 of a subcutaneous infusion set 14. Such infusion sets 14 are used to infuse medical fluids such as selected medications to a patient, with one example being the administration of insulin to a diabetic in the copending application 60/170,570 reusable injection delivery device using a dual mode injection system of a catheter or insulin pen tip for injection of medication.

After insertion of needle 12, the patient applies a section of adhesive tape to insure the needle is maintained safely in the skin and to maintain the base piece of the insertion set in position on the skin, when there is a base piece present with the needle.

FIG. 1 illustrates the details of the injector 10 with the slide deployed. The track 31 and the skin pucker 18 are visible in FIG. 1.

In use, the subcutaneous infusion set 14 can be placed quickly and easily into the sled 21 of the injector 10, within the recess formed in the injector and occupied by the sled 21. The catheter and needle 12 is retained in the sled by a small clip in the sled 21. Such assembly of the catheter 14 with the injector 10 requires simple alignment of the needle 12 and infusion tubing formed by 14 with the appropriate slot formed in the body of injector 10.

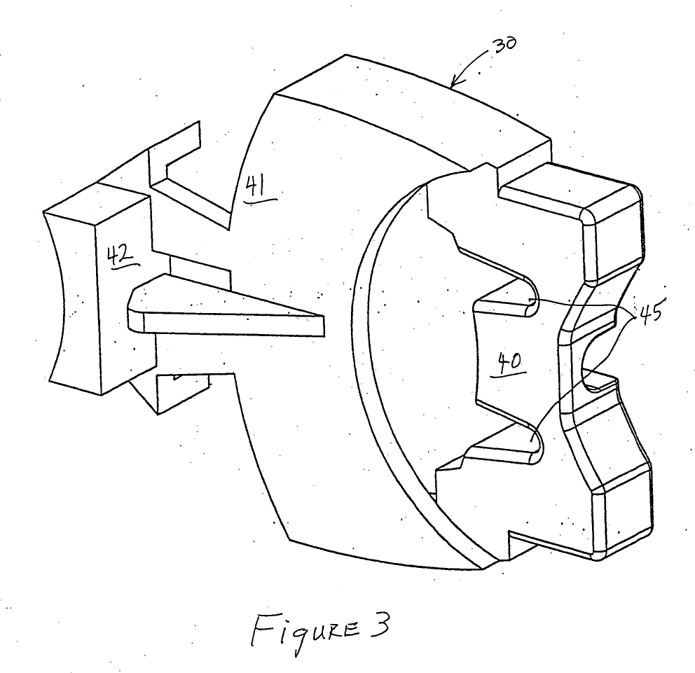

FIGS. 2, 2A and 3 illustrate embodiments of the adapter connector of this invention where first end 40 of the adapter 30 is configured to be inserted into and connected with the inserter device and the second end 41 of the adapter is configured to receive an insertion set that is not the correct size or shape to be compatible with the inserter device. Optionally and preferably the adapter includes retention latch 42, FIGS. 2 and 3, releasably securing the insertion set to the adapter during placement on the skin with the inserter device then releasing the insertion set from the adapter to remove the adapter and inserter device after placement is completed. Openings 45 are adapted for engaging retention members in the inserter device for releasably securing the adapter in the inserter device during use with an insertion set that is incompatible with the insertion device, but can be released to remove the adapter from the insertion device when used with an insertion set compatible with the inserter device without the adapter present.

The adapter connectors illustrated in FIGS. 2, 2A and 3 can be made of soft rubber or plastic, hard or rigid rubber or plastic, metal or other materials appropriate for the devices with which the adapter connectors will be used. A common material used in this type of device is a polycarbonate plastic. Soft rubber may be desired to produce friction fitting with the insertion device when latch 42 is not used and a configuration as show in FIG. 2A is used.

Other factors to be considered and as apparent to one skilled in the art for selection of materials and selection of particular configurations for the adapter connector include manufacturing cost, packaging and shipping convenience, consumer appeal and compatibility with the inserter devices and insertion sets of intended use.

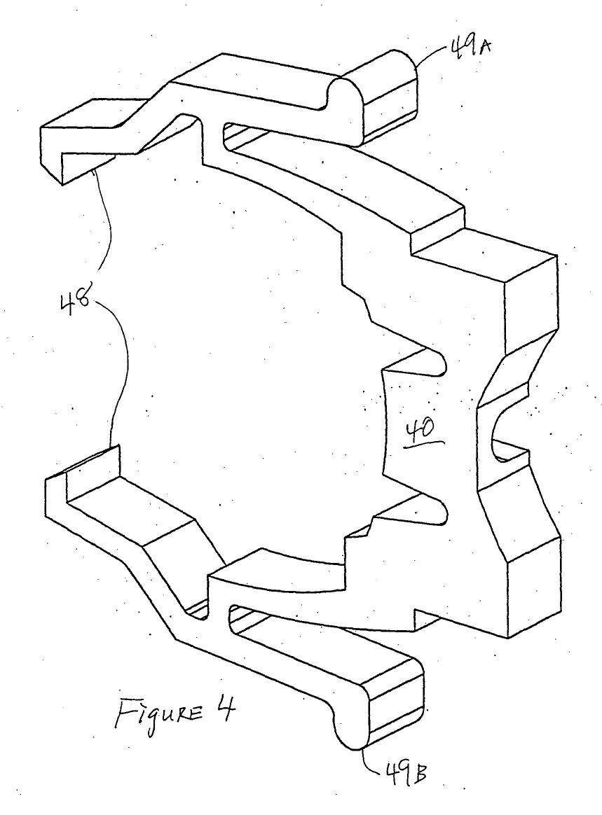

FIG. 4 illustrates a portion of an adapter connector that is adapted for the first end 40 to engage an inserter device and latches 48 to engage an insertion set of a different type. Grippers 49A and 49B are for use to disengage latches 48 from the insertion set.

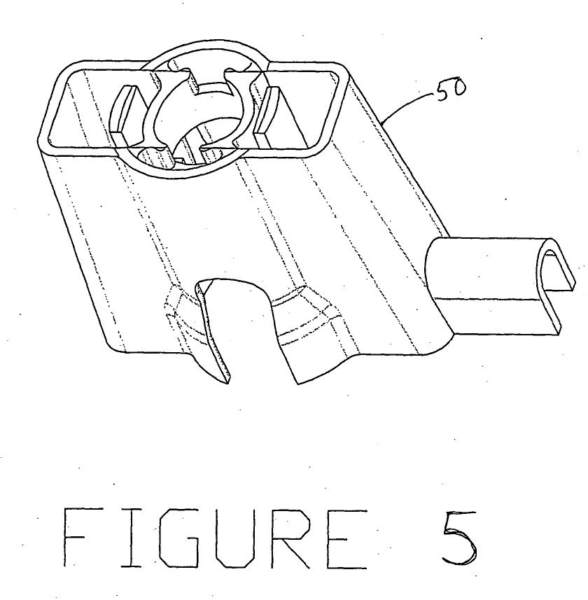

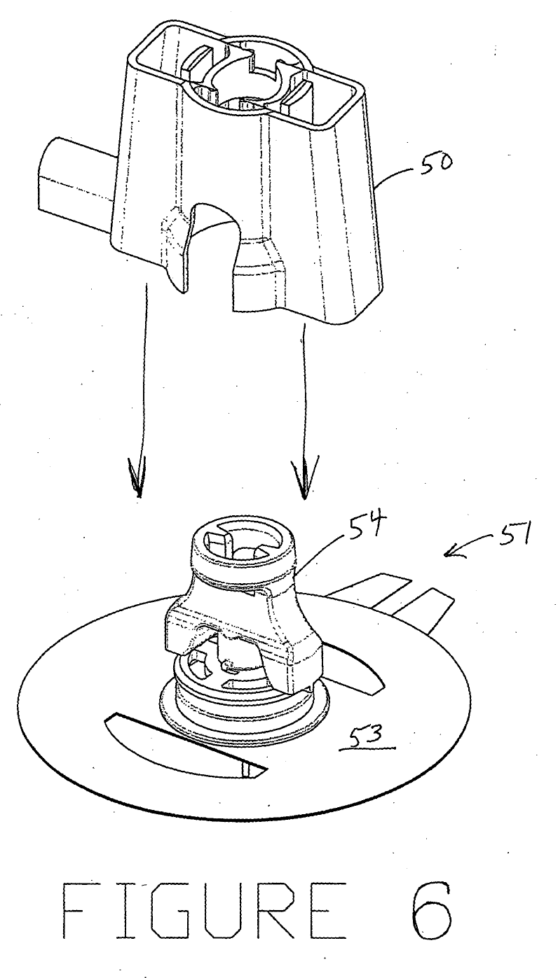

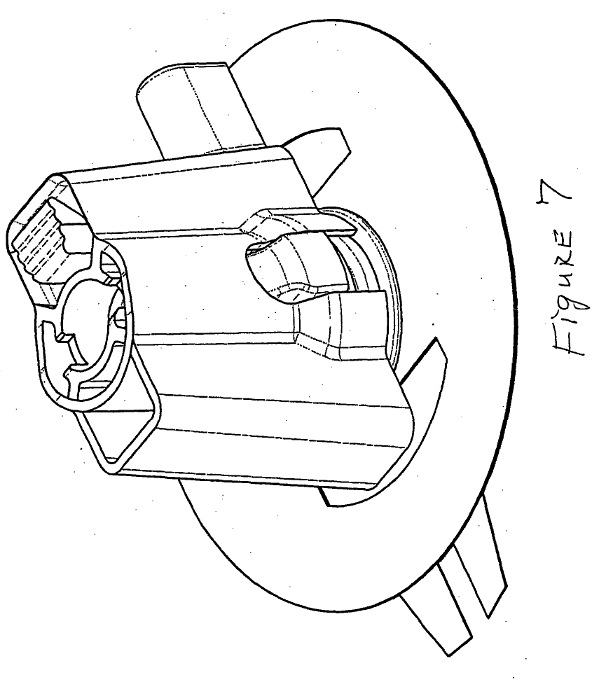

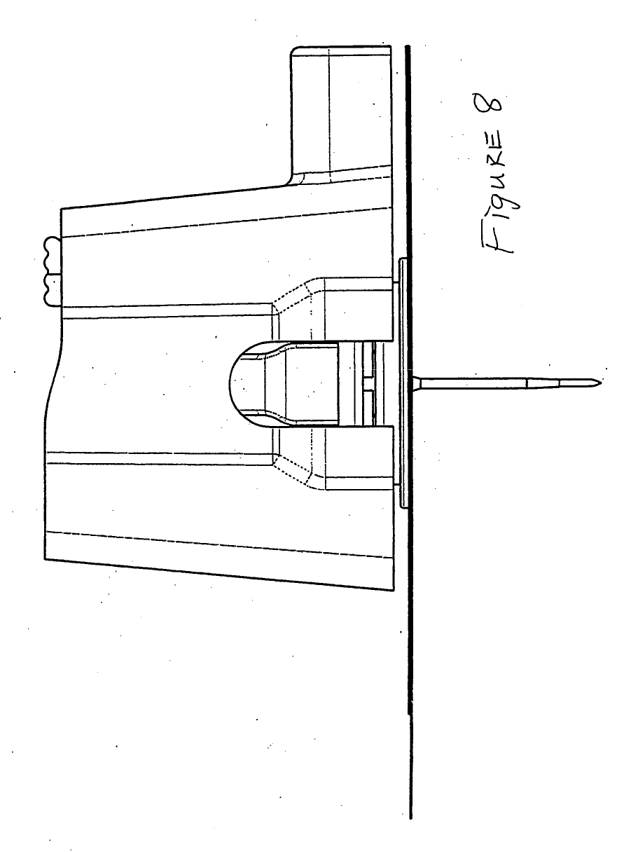

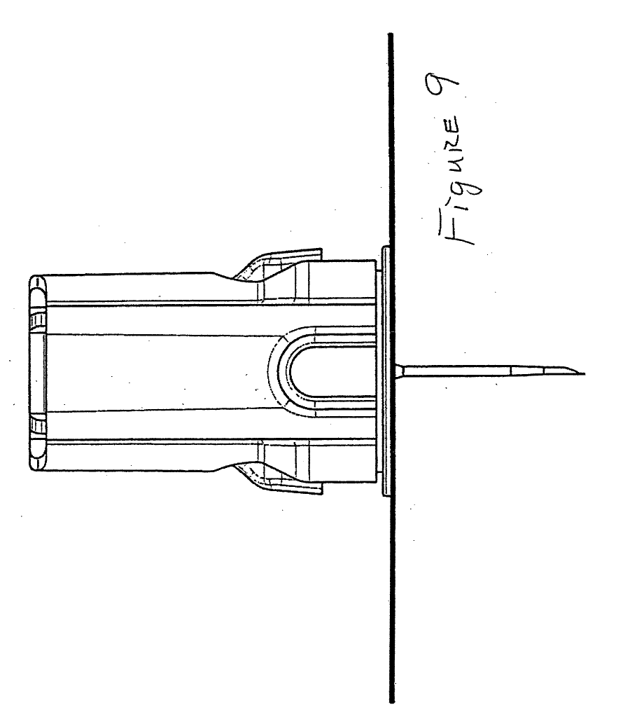

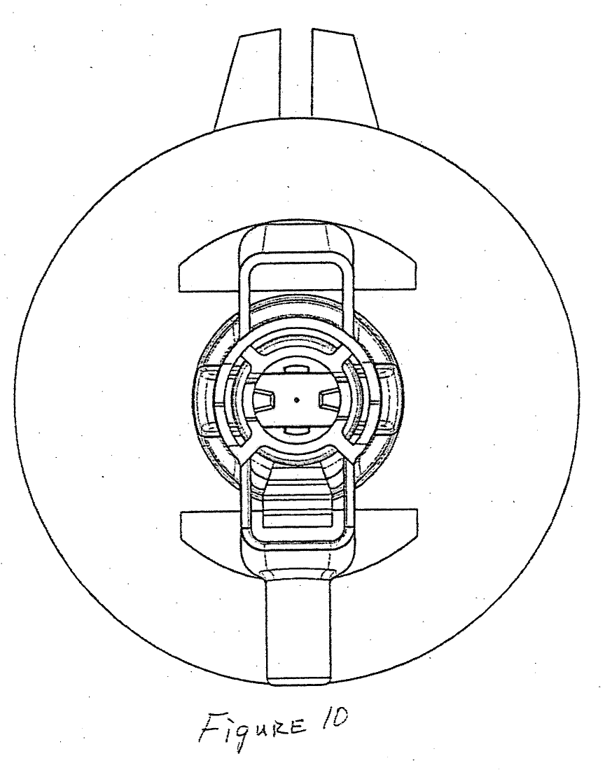

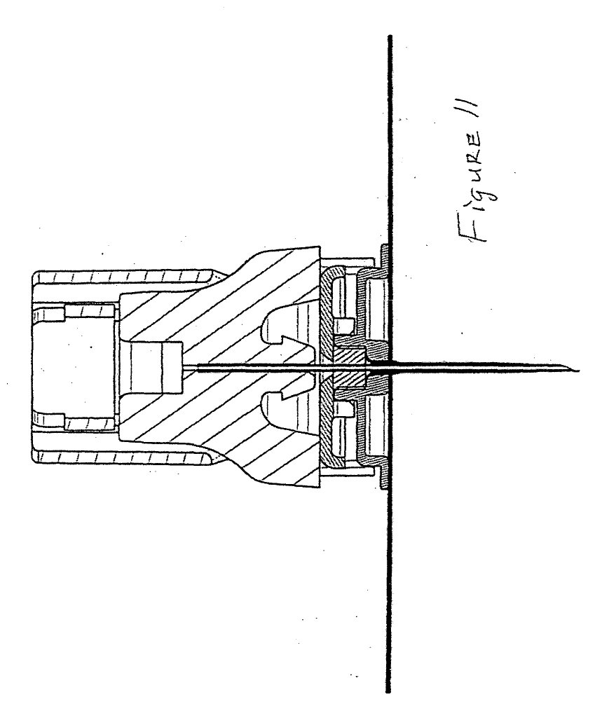

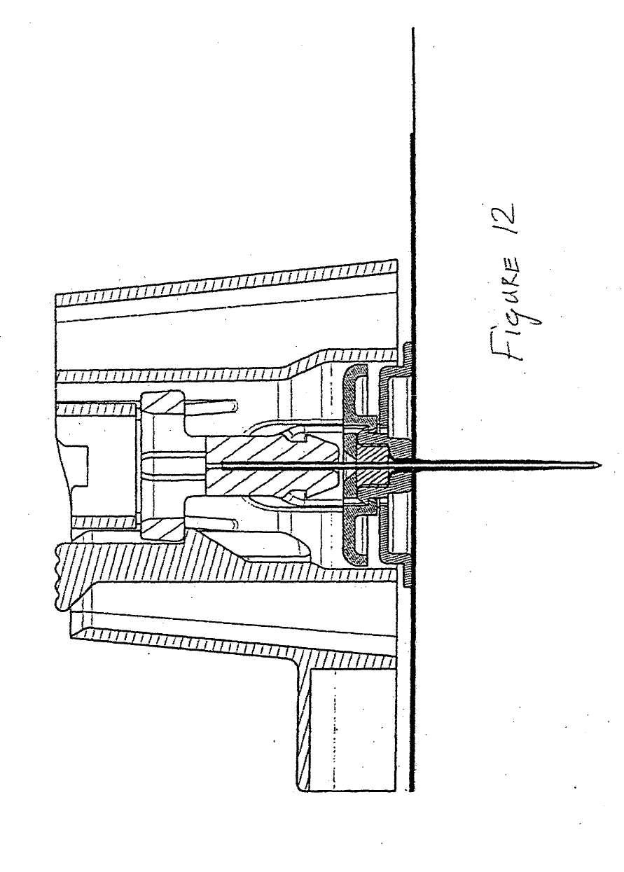

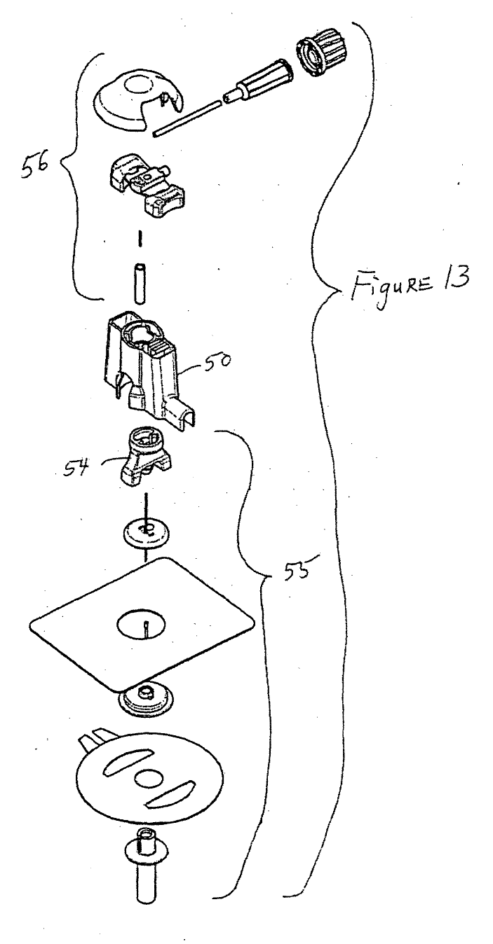

FIG. 5 illustrates an adapter connector 50 according to this invention configured for the vertical or 90° type of inserter device and insertion set 51, as illustrated in FIG. 6 wherein base piece 53 of the insertion set contacts the skin and removable piece 54 is replaced with the medication attachment after the infusion needle or cannula is in the correct subcutaneous position. FIGS. 7-12 show the adapter connector and insertion set in various views and sections. FIG. 13 is an exploded view of the system where the adapter connector is removed after placement of insertion set 55 and removable piece 54 is replaced with medication administration set 56.

The above disclosure enables one skilled in the art to implement this invention in any appropriate configuration and with any appropriate materials suitable for the devices and environment in which the invention will be used. The scope of the invention is set forth in the following claims.

What is claimed is:

1. An adapter connector for use in an insertion set and inserter system comprising a first end for connecting with the inserter and a second end for connecting with the insertion set wherein:
the first end of the connector is adapted to fit into and connect with the portion of the inserter device configured for receiving a two part insertion set having side positioned releasable latching means for holding the two parts of the insertion set together which latching means is located distal from the inserter device when the insertion set is positioned in the inserter device; and
the second end of the connector is adapted to receive a two-part insertion set having incompatible size or shape for fitting into the portion of the inserter device configured for receiving an insertion set and having side positioned releasable latching means for holding the two parts of the insertion set together which latching means is positioned proximal to the connector when the insertion set is positioned in the connector;
whereby the connector is adapted to connect the insertion set and the inserter, thereby enabling the use of the insertion set with the inserter device where the insertion set is otherwise incompatible with or unusable in said inserter device.

2. An adapter connector according to claim 1 wherein the adapter connector comprises openings in or near the first end adapted to receive and engage retention members in the inserter device for releasably holding the adapter connector in position in the inserter device.

3. An adapter connector according to claim 1 wherein the adapter connector comprises a latch on the second end adapted to engage and releasably hold the insertion set in position when engaged with the second end of the adapter connector.

4. An adapter connector according to claim 2 wherein the adapter connector comprises a latch on the second end adapted to engage and releasably hold the insertion set in position when engaged with the second end of the adapter connector.

5. An adapter connector according to claim 4 wherein the openings for engaging the retention members in the inserter device and the latch for holding the insertion set are relatively configured such that the insertion set is normally and more easily released from the adapter connector than the adapter connector is released from the inserter device whereby the adapter connector normally remains in the inserter device when the user releases the insertion set to remove the inserter device.

6. An adapter connector according to claim 1 wherein the adapter connector is configured for use in an inserter device that is adapted for use at an angle to the skin surface of between about 5 degrees and about 90 degrees from the skin surface.

7. An adapter connector according to claim 1 wherein the adapter connector is configured for use in an inserter device that is adapted for use at an angle to the skin surface of between about 20 degrees and about 45 degrees from the skin surface.

8. An adapter connector according to claim 1 wherein the adapter connector is configured for use in an inserter device that adapted for use at an angle of about 90 degrees from the skin surface.

9. A method for subcutaneous placement of an infusion needle or cannula with a system comprising an insertion set and an inserter device comprising the steps:
selecting an inserter device adapted for placement of an insertion set comprising an infusion needle or cannula;
selecting an insertion set that is incompatible with said inserter;
selecting an adapter connector having a first end adapted to fit into and connect with the portion of the inserter configured for receiving an insertion set and having a second end adapted to receive said insertion set that is incompatible by size or shape with said inserter device.

10. A method according to claim 9 comprising the steps:
placing the adapter connector in the inserter device; and
thereafter placing the insertion set in the adapter connector.

11. A method according to claim 9 comprising the steps:
placing the insertion set in the adapter connector; and
thereafter placing the adapter with the engaged insertion set in the inserter device.

12. A method according to claim 10 comprising the steps:
holding the inserter device at an angle between about 5 degrees and about 90 degrees from the skin surface;
releasing the inserter device sled to subcutaneously insert the infusion needle or cannula and place the insertion set on the skin surface;
and removing the adapter connector and inserter device from the insertion set.

13. A method according to claim 11 comprising the steps:
holding the inserter device at an angle between about 5 degrees and about 90 degrees from the skin surface;
releasing the inserter device to subcutaneously insert the infusion needle or cannula and place the insertion set on the skin surface;
and removing the adapter connector and inserter device from the insertion set.

14. A method according to claim 10 comprising the steps:
holding the inserter device at an angle between about 20 degrees and about 45 degrees from the skin surface;
releasing the inserter device sled to subcutaneously insert the infusion needle or cannula and place the insertion set on the skin surface;
and removing the adapter connector and inserter device from the insertion set.

15. A method according to claim 11 comprising the steps:
holding the inserter device at an angle between about 20 degrees and about 45 degrees from the skin surface;
releasing the inserter device to subcutaneously insert the infusion needle or cannula and place the insertion set on the skin surface;
and removing the adapter connector and inserter device from the insertion set.

16. A method according to claim 10 comprising the steps:
holding the inserter device at an angle of about 90 degrees from the skin surface;
releasing the inserter device to subcutaneously insert the infusion needle or cannula and place the insertion set on the skin surface;
and removing the adapter connector and inserter device from the insertion set.

17. A method according to claim 12 comprising the steps:
holding the inserter device at an angle of about 90 degrees from the skin surface;
releasing the inserter device to subcutaneously insert the infusion needle or cannula and place the insertion set on the skin surface;
and removing the adapter connector and inserter device from the insertion set.

18. A system for subcutaneous placement of an infusion needle or cannula comprising:
an inserter device adapted for subcutaneous placement of an infusion needle or cannula by placing an insertion set containing said needle or cannula against the surface of the skin;
an insertion set comprising an infusion needle or cannula adapted for subcutaneous placement having a size or shape incompatible for fitting into said inserter device; and
an adapter connector comprising a first end adapted to fit into the inserter device and a second end adapted to receive the insertion set;
whereby the adapter connector is adapted to connect the insertion set and the inserter device, thereby enabling the use of the insertion set with the inserter device where the insertion set is otherwise incompatible with or unusable in said inserter device.

19. A system according to claim 18 wherein the adapter connector comprises openings in or near the first end adapted to receive and engage retention members in the inserter device for releasably holding the adapter connector in position in the inserter device.

20. A system according to claim 18 wherein the adapter connector comprises a latch on the second end adapted to engage and releasably hold the insertion set in position when engaged with the second end of the adapter connector.

21. A system according to claim 19 wherein the adapter connector comprises a latch on the second end adapted to engage and releasably hold the insertion set in position when engaged with the second end of the adapter connector.

22. A system according to claim 21 wherein the openings for engaging the retention members in the inserter device and the latch for holding the insertion set are relatively configured such that the insertion set is normally more easily released from the adapter connector than the adapter connector is released from the inserter device whereby the adapter connector normally remains in the inserter device when the user releases the insertion set.

23. An adapter connector for use in an insertion set and inserter system comprising a first end for connecting with the inserter and a second end for connecting with the insertion set wherein:
the first end of the adapter connector is adapted to fit into and connect with the inserter device configured for receiving an insertion set comprising an infusion needle or cannula adapted for subcutaneous placement and
the second end of the adapter connector is adapted to receive an insertion set having incompatible size or shape for fitting into the inserter device;
whereby the adapter connector is adapted to connect the insertion set and the inserter, thereby enabling the use of the insertion set with the inserter device where the insertion set is otherwise incompatible with or unusable in said inserter device.

24. An adapter connector according to claim 23 wherein the adapter connector comprises openings in or near the first end adapted to receive and engage retention members in the inserter device for releasably holding the adapter connector in position in the inserter device.

25. An adapter connector according to claim 23 wherein the adapter connector comprises a latch on the second end adapted to engage and releasably hold the insertion set in position when engaged with the second end of the adapter connector.

26. An adapter connector according to claim 24 wherein the adapter connector comprises a latch on the second end adapted to engage and releasably hold the insertion set in position when engaged with the second end of the adapter connector.

27. An adapter connector according to claim 26 wherein the openings for engaging the retention members in the inserter device and the latch for holding the insertion set are relatively configured such that the insertion set is preferentially and more easily released from the adapter connector than the adapter connector is released from the inserter device whereby the adapter connector preferentially remains in the inserter device when the user releases the insertion set.

28. An adapter connector for use in an insertion set and inserter system comprising a first end for connecting with the inserter and a second end for connecting with the insertion set wherein:
the first end of the adapter connector containing means for fitting into and connecting with the inserter device configured for receiving an insertion set comprising an infusion needle or cannula adapted for subcutaneous placement and
the second end of the adapter connector containing means for receiving an insertion set having incompatible size or shape for fitting into the inserter device;
whereby the adapter connector contains means for connecting the insertion set and the inserter, thereby enabling the use of the insertion set with the inserter device where the insertion set is otherwise incompatible with or unusable in said inserter device.

29. An adapter connector according to claim 28 wherein the adapter connector comprises means in or near the first end adapted for receiving and engaging retention members in the inserter device for releasably holding the adapter connector in position in the inserter device.

30. An adapter connector according to claim 29 wherein the adapter connector comprises a latch means on the second end adapted for engaging and releasably holding the insertion set in position when engaged with the second end of the adapter connector.
